(19) United States
(12) Patent Application Publication
(10) Pub. No.: US 2024/0266048 A1
(43) Pub. Date: Aug. 8, 2024

(54) DIAGNOSIS DEVICE PROVIDING DIAGNOSIS INFORMATION AND DIAGNOSIS BASIS INFORMATION, AND METHOD OF OPERATING THE SAME

(71) Applicant: ELECTRONICS AND TELECOMMUNICATIONS RESEARCH INSTITUTE, Daejeon (KR)

(72) Inventors: Sungyup LEE, Daejeon (KR); Yongjin LEE, Daejeon (KR)

(21) Appl. No.: 18/419,800

(22) Filed: Jan. 23, 2024

(30) Foreign Application Priority Data
Feb. 3, 2023 (KR) 10-2023-0015107

(51) Int. Cl. G16H 50/20; A61B 5/08; A61B 7/00

(52) U.S. Cl. CPC G16H 50/20; A61B 5/0816; A61B 7/003

(57) ABSTRACT

Disclosed is a method of operating a diagnosis device for diagnosing a disease of a user, which includes obtaining breathing sound data by measuring a breath of the user, generating feature parameter information including a plurality of parameters corresponding to at least one abnormal breathing sound based on an analysis of the breathing sound data, generating diagnosis information indicating a target disease with a highest model output value among a plurality of diseases by applying the feature parameter information to a pre-trained differential diagnosis model, generating diagnosis basis information that quantifies an importance of the plurality of parameters used to determine the target disease, and outputting the diagnosis information and the diagnosis basis information through a user interface device of the diagnosis device.

### CROSS-REFERENCE TO RELATED APPLICATION(S)

This application claims priority under 35 U.S.C. § 119 to Korean Patent Application No. 10-2023-0015107 filed on Feb. 3, 2023, in the Korean Intellectual Property Office, the disclosures of which are incorporated by reference herein in their entireties.

### BACKGROUND

### 1. Field of the Invention

Embodiments of the present disclosure described herein relate to a diagnosis device, and more particularly, relate to a diagnosis device that provides diagnosis information and diagnosis basis information, and a method of operating the same.

### 2. Description of Related Art

Abnormal breathing sounds of a patient are an important factor in diagnosing a disease of the patient. A disease diagnosis model using a deep neural network may detect abnormal breathing sounds from breathing sounds of the patient and may classify diseases based on the abnormal breathing sounds, but does not provide a detail basis that classifies the disease. Therefore, there is a problem that it is difficult for professional organizations such as medical institutions to trust the disease classification results despite the accuracy of the disease diagnosis models.

Accordingly, to classify the disease of a patient based on the breathing sounds of the patient and explain the basis for disease classification, there may be a need for a diagnosis device that may provide information in a form that doctors may understand by using the importance of the features of abnormal breathing sounds used to diagnose the disease as the basis for disease diagnosis.

### SUMMARY

Embodiments of the present disclosure provide a diagnosis device that provides diagnosis information and diagnosis basis information and a method of operating the same.

According to an embodiment of the present disclosure, a method of operating a diagnosis device for diagnosing a disease of a user includes obtaining breathing sound data by measuring a breath of the user, generating feature parameter information including a plurality of parameters corresponding to at least one abnormal breathing sound based on an analysis of the breathing sound data, generating diagnosis information indicating a target disease with a highest model output value among a plurality of diseases by applying the feature parameter information to a pre-trained differential diagnosis model, generating diagnosis basis information that quantifies an importance of the plurality of parameters used to determine the target disease, and outputting the diagnosis information and the diagnosis basis information through a user interface device of the diagnosis device.

According to an embodiment, the generating of the diagnosis information indicating a target disease with the highest model output value among the plurality of diseases by applying the feature parameter information to the pre-trained differential diagnosis model may include observing a change in an output of the pre-trained differential diagnosis model depending on a change in at least one parameter among the plurality of parameters of the feature parameter information, calculating the importance of the plurality of parameters depending on the change in the output of the pre-trained differential diagnosis model, and generating the diagnosis information indicating the target disease based on the importance of the plurality of parameters.

According to an embodiment, the importance of the plurality of parameters may indicate a priority of each of the plurality of parameters with respect to the target disease.

According to an embodiment, the plurality of parameters of the feature parameter information may include at least one of the number of occurrences, occurrence intensity, start time, end time, duration, lowest frequency, maximum frequency, and average frequency of the at least one abnormal breathing sound.

According to an embodiment, the generating of the feature parameter information including the plurality of parameters corresponding to the at least one abnormal breathing sound based on the analysis of the breathing sound data may include generating spectrogram data that visually represents a time component and a frequency component of the at least one abnormal breathing sound based on the breathing sound data, generating heatmap data including pixels each having a temperature value depending on a probability of corresponding to the at least one abnormal breathing sound based on a convolution neural network operation of the spectrogram data, and generating the feature parameter information based on the spectrogram data and the heatmap data.

According to an embodiment, the generating of the feature parameter information based on the spectrogram data and the heatmap data may include detecting a blob region by filtering the heatmap data based on a threshold temperature value, extracting coordinates of the blob region from the heatmap data, and generating the feature parameter information corresponding to the time component and the frequency component of the at least one abnormal breathing sound based on the spectrogram data and the extracted coordinates.

According to an embodiment, the generating of the feature parameter information corresponding to the time component and the frequency component of the at least one abnormal breathing sound based on the spectrogram data and the extracted coordinates may include calculating the plurality of parameters corresponding to the time component based on abscissa coordinate values of the extracted coordinates and a reference time length, and the reference time length may be determined based on equation

$${t}_{p} = \frac{{S}_{rate}}{{L}_{hop}} ,$$

where tₚ refers to the reference time length, Sᵣₐₜₑ refers to a sampling rate of the breathing sound data, and Lₕₒₚ refers to a hop length of the spectrogram data, and the plurality of parameters corresponding to the time component are determined based on equation tₓ=bₓ×tₚ, where tₚ indicates the reference time length, bₓ indicates one of the abscissa coordinate values, and tₓ indicates one of the plurality of parameters corresponding to the time component.

According to an embodiment, the generating of the feature parameter information corresponding to the time component and the frequency component of the at least one abnormal breathing sound based on the spectrogram data and the extracted coordinates may include calculating a plurality of parameters corresponding to the frequency component based on ordinate coordinate values of the extracted coordinates and a reference frequency magnitude, and the reference frequency magnitude may be determined based on equation

$${f}_{p} = \frac{{S}_{rate}}{{N}_{f}} ,$$

fₚ refers to the reference frequency magnitude, Sᵣₐₜₑ refers to a sampling rate of the breathing sound data, and N_{f} refers to the number of reference samples, and the plurality of parameters corresponding to the frequency component may be determined based on equation f_{y}=b_{y}+fₚ, where fₚ indicates the reference frequency magnitude, b_{y} indicates one of the ordinate coordinate values, and f_{y} indicates one of the plurality of parameters corresponding to the frequency component.

According to an embodiment, the generating of the heatmap data including pixels each having the temperature value depending on the probability of corresponding to the at least one abnormal breathing sound based on a convolution neural network operation of the spectrogram data may include generating the heatmap data from the spectrogram data based on a top-down method.

According to an embodiment of the present disclosure, a diagnosis device includes a sound sensor that measures a breath of a user to generate breathing sound data, a breathing sound analysis device that generates spectrogram data that visually represents a time component and a frequency component of at least one abnormal breathing sound based on an analysis of the breathing sound data, and generates heatmap data including pixels each having a temperature value depending on a probability of corresponding to the at least one abnormal breathing sound based on a convolution neural network operation of the spectrogram data, a feature parameter extraction device that generates feature parameter information including a plurality of parameters corresponding to the at least one abnormal breathing sound based on the spectrogram data and the heatmap data, a differential diagnosis device that generates diagnosis information indicating a target disease with a highest model output value among a plurality of diseases by applying the feature parameter information to a pre-trained differential diagnosis model, and generates diagnosis basis information that quantifies an importance of the plurality of parameters used to determine the target disease, and a user interface device that outputs the diagnosis information and the diagnosis basis information.

According to an embodiment, a breathing sound analysis device may include a spectrogram generator that receives the breathing sound data from the sound sensor and generates the spectrogram data from the breathing sound data, and an abnormal breathing sound detection device that receives the spectrogram data from the spectrogram generator and generates the heatmap data from the spectrogram data.

According to an embodiment, the feature parameter extraction device may include a blob region detector that receives the heatmap data from the abnormal breathing sound detection device, detects a blob region by filtering the heatmap data based on a threshold temperature value, extracts coordinates of the blob region from the heatmap data, and generates blob region information indicating the extracted coordinates, and a feature parameter generator that receives the spectrogram data from the spectrogram generator, receives the blob region information from the blob region detector, and generates the feature parameter information corresponding to the time component and the frequency component of the at least one abnormal breathing sound, based on the spectrogram data and the blob region information.

According to an embodiment, the differential diagnosis device may include a diagnosis information generator that receives the feature parameter information from the feature parameter extraction device, observes a change in an output of the pre-trained differential diagnosis model depending on a change in at least one parameter among the plurality of parameters of the feature parameter information, calculates the importance of the plurality of parameters depending on the change in the output of the pre-trained differential diagnosis model, and generates the diagnosis information indicating the target disease based on the importance of the plurality of parameters, and a diagnosis basis information generator that generates the diagnosis basis information that quantifies the importance of the plurality of parameters used to determine the target disease.

### BRIEF DESCRIPTION OF THE DRAWINGS

The above and other objects and features of the present disclosure will become apparent by describing in detail embodiments thereof with reference to the accompanying drawings.

FIG. 1 is a block diagram illustrating a diagnosis device according to an embodiment of the present disclosure.

FIG. 2 is a diagram illustrating a breathing sound analysis device of FIG. 1 according to some embodiments of the present disclosure.

FIG. 3 is a diagram illustrating a feature parameter extraction device of FIG. 1 according to some embodiments of the present disclosure.

FIG. 4 is a diagram illustrating a differential diagnosis device of FIG. 1 according to some embodiments of the present disclosure.

FIG. 5 is a diagram illustrating blob region information according to some embodiments of the present disclosure.

FIG. 6 is a flowchart describing a method of operating a diagnosis device according to some embodiments of the present disclosure.

FIG. 7 is a flowchart describing a method of generating diagnosis information by a differential diagnosis device according to some embodiments of the present disclosure.

### DETAILED DESCRIPTION

Hereinafter, embodiments of the present disclosure will be described in detail and clearly to such an extent that an ordinary one in the art easily implements the present disclosure.

The function blocks illustrated in drawings used below may be implemented in the form of a software component, a hardware component, or a combination thereof. Below, to describe the technical idea of the inventive concept clearly, a description associated with identical components will be omitted.

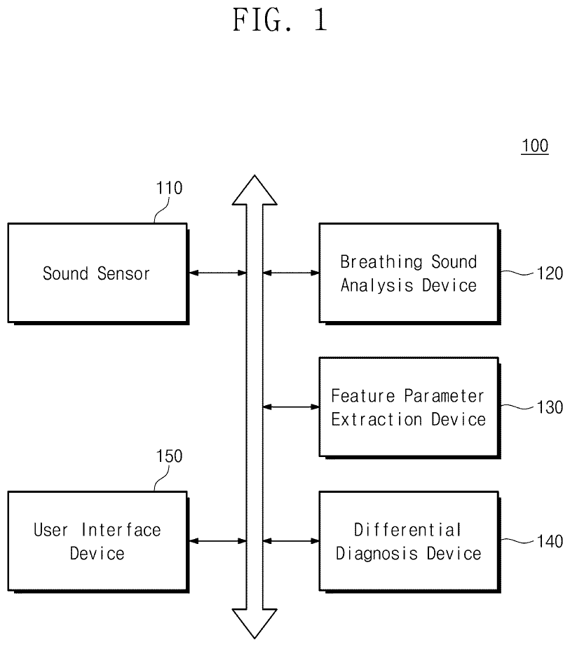

FIG. 1 is a block diagram illustrating a diagnosis device according to an embodiment of the present disclosure. Referring to FIG. 1, a diagnosis device 100 is illustrated. The diagnosis device 100 may be a device that generates disease diagnosis information and diagnosis basis information of users and provides the information to the users through a user interface device. The user may be a person who is breathing and has a respiratory disease or is suspected of having a respiratory disease. For example, the diagnosis device 100 may be implemented as one of various electronic devices that analyze breathing sounds, such as a smart phone, tablet personal computer (PC), desktop, PC, and laptop.

The diagnosis device 100 may include a sound sensor 110, a breathing sound analysis device 120, a feature parameter extraction device 130, a differential diagnosis device 140, and a user interface device 150. For example, the sound sensor 110 and the user interface device 150 may be configured to be included in electronic devices such as smart phones and tablet PCs, and the breathing sound analysis device 120, the feature parameter extraction device 130, and the differential diagnosis device 140 may be a server that communicates with smart phones, tablet PCs, etc.

The sound sensor 110 may obtain breathing sound data by measuring the user's breathing. The sound sensor 110 may measure the user's breathing for a specific period of time. The breathing sound data may be sound data corresponding to the user's breathing sound. The sound sensor 110 may provide the breathing sound data to the breathing sound analysis device 120.

The breathing sound analysis device 120 may receive breathing sound data from the sound sensor 110. The breathing sound analysis device 120 may analyze the breathing sound data and may generate image data corresponding to at least one abnormal breathing sound. The abnormal breathing sound may refer to a breathing sound that is correlated with the user's disease. The abnormal breathing sound may have time features above a threshold value or frequency features above a threshold value.

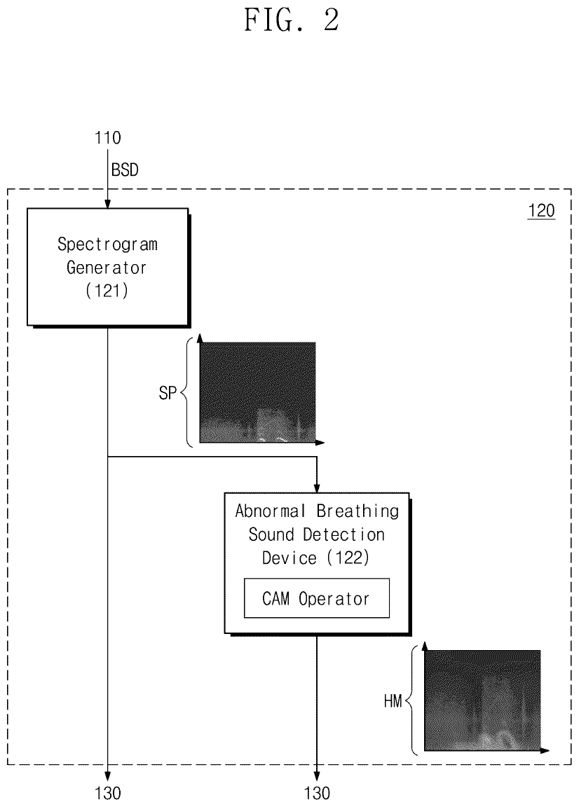

The breathing sound analysis device 120 may generate spectrogram data based on the breathing sound data. The spectrogram data may be image data that visually represents a time component and frequency component of at least one abnormal breathing sound. A more detailed description of the spectrogram data will be described later with reference to FIG. 2.

The breathing sound analysis device 120 may generate heatmap data based on the spectrogram data. The heatmap data may be image data including pixels each having a temperature value according to the probability of corresponding to at least one abnormal breathing sound based on a convolution neural network (CNN) operation of the spectrogram data. A more detailed description of the heatmap data will be described later with reference to FIG. 2.

The breathing sound analysis device 120 may provide the spectrogram data and the heatmap data to the feature parameter extraction device 130.

The feature parameter extraction device 130 may receive the spectrogram data and the heatmap data from the breathing sound analysis device 120. The feature parameter extraction device 130 may generate feature parameter information including a plurality of parameters corresponding to at least one abnormal breathing sound based on the spectrogram data and the heatmap data. For example, the plurality of parameters may include at least one of the number of occurrences, intensity of occurrence, start time, end time, duration, lowest frequency, maximum frequency, and average frequency of at least one abnormal breathing sound. The feature parameter extraction device 130 may provide feature parameter information to the differential diagnosis device 140.

The differential diagnosis device 140 may receive feature parameter information from the feature parameter extraction device 130. The differential diagnosis device 140 may generate diagnosis information and diagnosis basis information by applying the feature parameter information to a pre-trained diagnosis model. The pre-trained diagnosis model may be a machine learning model that includes neural network operations. The neural network operations may include learning and inference operations. The pre-trained diagnosis model may be obtained by training based on training data in a learning stage prior to disease inference through the differential diagnosis device 140. For example, the training data may be large-scale data representing disease-breathing sounds.

The diagnosis information may indicate a target disease with the highest model output value among a plurality of diseases in a pre-trained diagnosis model. The model output value may represent the likelihood (e.g., probability) of corresponding to the corresponding disease.

The diagnosis basis information may be information that quantifies the importance of each of a plurality of parameters used to determine the target disease.

In some embodiments, the importance of the plurality of parameters may indicate the priority of each of the plurality of parameters with respect to the target disease.

The differential diagnosis device 140 may provide the diagnosis information and the diagnosis basis information to the user interface device 150.

The user interface device 150 may receive the diagnosis information and the diagnosis basis information from the differential diagnosis device 140. The user interface device 150 may provide the diagnosis information and the diagnosis basis information to the user.

FIG. 2 is a diagram illustrating a breathing sound analysis device of FIG. 1 according to some embodiments of the present disclosure. Referring to FIG. 2, the breathing sound analysis device 120 is illustrated.

The breathing sound analysis device 120 may include a spectrogram generator 121 and an abnormal breathing sound detection device 122.

The spectrogram generator 121 may receive breathing sound data BSD from the sound sensor 110. The spectrogram generator 121 may generate spectrogram data SP from breathing sound data BSD.

The spectrogram data SP is image data in the form of a graph, where a horizontal axis may indicate time and a vertical axis may indicate frequency. The value of each pixel of the spectrogram data SP may indicate the intensity of the breathing sound corresponding to the time and frequency of each pixel. For example, a pixel may have a light color when the intensity of the breathing sound corresponding to the time and frequency of the pixel is large, and a pixel may have a dark color when the intensity of the breathing sound corresponding to the time and frequency of the pixel is small.

The spectrogram generator 121 may provide the spectrogram data SP to the abnormal breathing sound detection device 122 and the feature parameter extraction device 130.

The abnormal breathing sound detection device 122 may receive the spectrogram data SP from the spectrogram generator 121. The abnormal breathing sound detection device 122 may detect abnormal breathing sounds by applying a convolution neural network to the spectrogram data.

The abnormal breathing sound detection device 122 may include a CAM operator. The CAM operator may generate heatmap data HM by presenting elements that are the basis for the convolution neural network to detect abnormal breathing sounds. Each pixel of the heatmap data HM may have a high temperature value when the probability of corresponding to at least one abnormal breathing sound is high, and may have a low temperature value when the probability of corresponding to at least one abnormal breathing sound is low. For example, pixels with high temperature values are displayed in red and may be referred to as a hot-region. Pixels with low temperature values are displayed in blue and may be referred to as a cold-region.

In some embodiments, the heatmap data HM may refer to a top-down heatmap. The CAM operator may generate a top-down heatmap based on the top-down method.

Generally, a neural network includes a plurality of layers, and may perform a neural network operation by sequentially applying the plurality of layers to image data from a low layer to a high layer. As the layer is lower, the layer is closer to an input of the neural network, and a size of the filter data may be larger. As the layer is higher, the layer is closer to an output of the neural network, and a size of the filter data may be smaller.

As the layer is lower, the resolution of the heatmap generated based on the layer is higher, but the accuracy of object discrimination may be lower. In contrast, as the layer is higher, the resolution of the heatmap generated based on the layer is lower, but the accuracy of object discrimination may be higher.

The top-down method may be a method of generating a high-resolution heatmap with high accuracy in object discrimination by applying the plurality of layers to image data in reverse order. In detail, the top-down method may be a method of sequentially applying the plurality of layers to image data in direction from a high layer to a low layer.

The abnormal breathing sound detection device 122 may provide the heatmap data HM to the feature parameter extraction device 130.

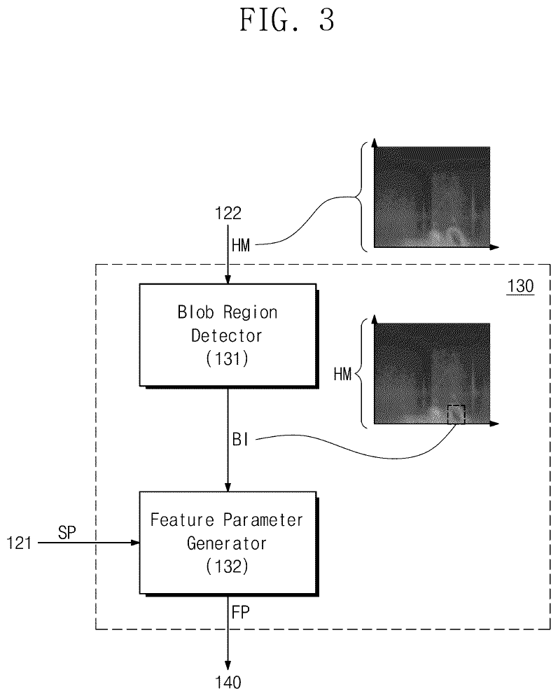

FIG. 3 is a diagram illustrating a feature parameter extraction device of FIG. 1 according to some embodiments of the present disclosure. Referring to FIG. 3, the feature parameter extraction device 130 is illustrated.

The feature parameter extraction device 130 may include a blob region detector 131 and a feature parameter generator 132.

The blob region detector 131 may receive the heatmap data HM from the abnormal breathing sound detection device 122. The blob region detector 131 may detect the blob region by filtering the heatmap data HM based on a threshold temperature value. The threshold temperature value may be set by the user. The blob region is a region corresponding to at least one abnormal breathing sound and may be a region including pixels with a temperature value higher than the threshold temperature value. In detail, the blob region may include a hot-region of the heatmap data HM.

Referring to FIG. 3, the heatmap data HM may include blob region information. Although the blob region information is displayed as a rectangular region, the scope of the present disclosure is not limited thereto.

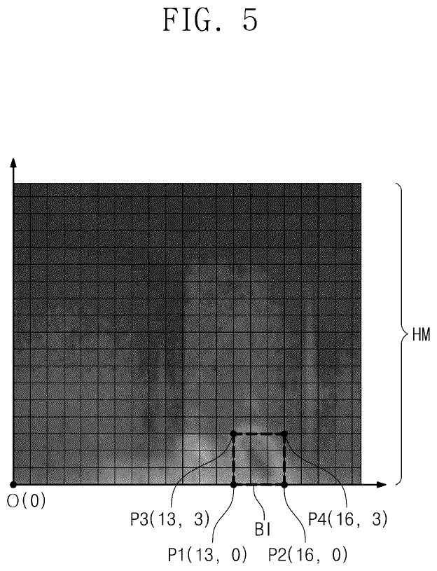

The blob region detector 131 may generate blob region information BI by extracting coordinates of the blob region from the heatmap data HM. The blob region information BI is the location of the blob region in the heatmap data HM and may indicate the coordinates of the blob region. The coordinates of the blob region may include coordinates of each vertex of the blob region and coordinates within the blob region. A more detailed description of the blob region information BI will be described later with reference to FIG. 5.

The blob region detector 131 may provide blob region information BI to the feature parameter generator 132.

The feature parameter generator 132 may receive the spectrogram data SP from the spectrogram generator 121 and may receive the blob region information BI from the blob region detector 131. The feature parameter generator 132 may generate feature parameter information FP corresponding to the time component and frequency component of at least one abnormal breathing sound based on the spectrogram data SP and the blob region information BI. The feature parameter information FP may include a plurality of parameters indicating the time component and frequency component of at least one abnormal breathing sound. Each of the plurality of parameters may also be referred to as a feature parameter.

The feature parameter generator 132 may generate the feature parameter information FP corresponding to the time component of at least one abnormal breathing sound by applying the abscissa coordinate values of the blob region information BI to the spectrogram data SP. For example, the feature parameter information FP corresponding to the time component may include at least one of the number of occurrences, start time, end time, and duration of the abnormal breathing sound.

The feature parameter generator 132 may generate the feature parameter information FP corresponding to the frequency component of at least one abnormal breathing sound by applying the ordinate coordinate values of the blob region information BI to the spectrogram data SP. For example, the feature parameter information FP corresponding to the frequency component may include at least one of the occurrence intensity, lowest frequency, maximum frequency, and average frequency of the abnormal breathing sound.

A more detailed description of the process of generating feature parameter information FP will be described later with reference to FIG. 5.

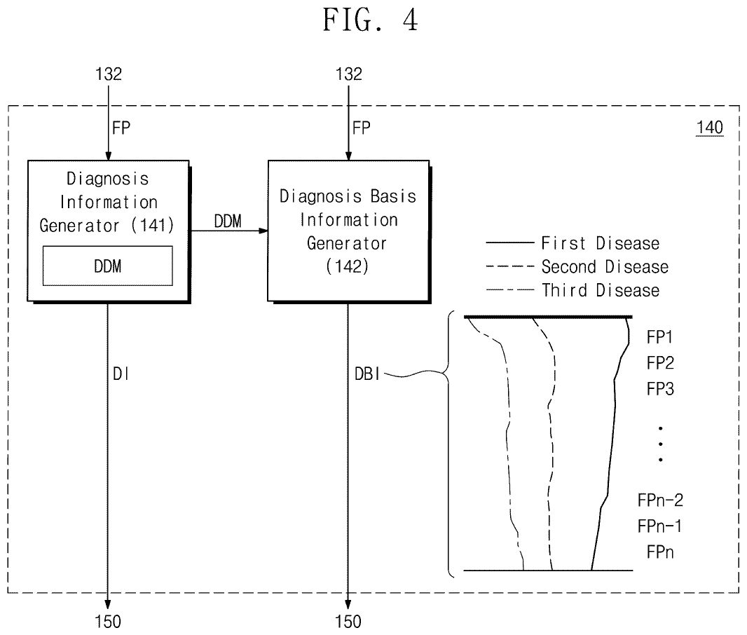

FIG. 4 is a diagram illustrating a differential diagnosis device of FIG. 1 according to some embodiments of the present disclosure. Referring to FIG. 4, the differential diagnosis device 140 and diagnosis basis information DBI are illustrated.

The differential diagnosis device 140 may include a diagnosis information generator 141 and a diagnosis basis information generator 142.

The diagnosis information generator 141 may receive the feature parameter information FP from the feature parameter generator 132. The diagnosis information generator 141 may include a pre-trained differential diagnosis model DDM. The pre-trained differential diagnosis model DDM may be a machine learning model that receives a plurality of parameters of the feature parameter information FP as input and outputs one of a plurality of diseases. The diagnosis information generator 141 may generate diagnosis information DI indicating the target disease with the highest model output value among the plurality of diseases by applying the feature parameter information FP to the pre-trained differential diagnosis model DDM.

In some embodiments, the diagnosis information generator 141 may observe a change in the output of the pre-trained differential diagnosis model DDM according to a change in at least one parameter among the plurality of parameters of the feature parameter information FP. For example, the output of the pre-trained differential diagnosis model DDM when one parameter among the plurality of parameters is present may be compared with the output of the pre-trained differential diagnosis model DDM when one parameter among the plurality of parameters is absent.

The diagnosis information generator 141 may calculate the importance of the plurality of parameters according to changes in the output of the pre-trained differential diagnosis model DDM. In addition, the diagnosis information generator 141 may generate the diagnosis information DI indicating the target disease based on the importance of the plurality of parameters.

The diagnosis information generator 141 may provide the diagnosis information DI to the user interface device 150. The diagnosis information generator 141 may transfer the pre-trained differential diagnosis model DDM to the diagnosis basis information generator 142.

The diagnosis basis information generator 142 may receive the feature parameter information FP from the feature parameter generator 132 and may receive the pre-trained differential diagnosis model DDM from the diagnosis information generator 141.

The diagnosis basis information generator 142 may generate the diagnosis basis information DBI by quantifying the importance of the plurality of parameters used to determine the target disease, based on the feature parameter information FP and the pre-learned differential diagnosis model DDM.

In some embodiments, the diagnosis basis information DBI may quantify and represent the importance of each of the plurality of parameters for the disease diagnosis with respect to each of a plurality of diseases. For example, the diagnosis basis information DBI may indicate the importance of each of first to n-th parameters FP1 to FPn used to determine one of first to third diseases as the target disease. A vertical axis may indicate the first to n-th parameters FP1 to FPn, and a horizontal axis may indicate quantified importance.

In the diagnosis basis information DBI, the solid line may indicate the first disease. In the diagnosis basis information DBI, the dotted line may indicate the second disease. In the diagnosis basis information DBI, the dashed-dot line may indicate the third disease. However, the scope of the present disclosure is not limited thereto.

For example, regarding the pre-trained diagnostic model DDM determining the first disease as the target disease, the diagnosis basis information DBI may indicate the first parameter FP1, the second parameter FP2, and the third parameter FP3 in order of quantified importance as the basis diagnosing the first disease.

For example, regarding the pre-trained diagnostic model DDM determining the second disease as the target disease, the diagnosis basis information DBI may indicate the third parameter FP3 and the second parameter FP2 in order of quantified importance as the basis diagnosing the second disease.

For example, regarding the pre-trained diagnostic model DDM determining the third disease as the target disease, the diagnosis basis information DBI may indicate the n-th parameter FPn and the (n−1)-th FPn-1 in order of quantified importance as the basis diagnosing the third disease.

The diagnosis basis information generator 142 may provide the diagnosis basis information DBI to the user interface device 150.

FIG. 5 is a diagram illustrating blob region information according to some embodiments of the present disclosure. Referring to FIG. 5, the blob region information BI will be described. The heatmap data HM and the blob region information BI of FIG. 5 may correspond to the heatmap data HM and the blob region information BI of FIG. 3.

The blob region detector 131 may set coordinate axes to the heatmap data HM and may calculate coordinate values included in the blob region. The horizontal axis set in the heatmap data HM may correspond to the time features of the blob region, and the vertical axis set in the heatmap data HM may correspond to the frequency features of the blob region.

For example, the blob region information may include the coordinates of each of the first to fourth points P1 to P4, which are vertices of the blob region information.

The horizontal axis coordinate of any one point in the blob region information may indicate the number of pixels in the horizontal direction between an origin ‘O’ and a point. The vertical axis coordinate of any one point in the blob region information may indicate the number of pixels in the vertical direction between the origin ‘O’ and a point. A size of one pixel of the heatmap data HM may be the same as a size of one pixel of spectrogram data having the same size as the heatmap data HM. The size of one pixel of the spectrogram data may be determined based on a reference time length and a reference frequency length, and a more detailed description of this will be provided later.

For example, the horizontal axis coordinate of a first point P1 may be 13, which is the number of pixels in the horizontal direction between the origin O and the first point P1. The vertical axis coordinate of the first point P1 may be ‘0’, which is the number of pixels in the vertical direction between the origin ‘O’ and the first point P1.

For example, the horizontal axis coordinate of a second point P2 may be 16, which is the number of pixels in the horizontal direction between the origin O and the second point P2. The vertical axis coordinate of the second point P2 may be ‘0’, which is the number of pixels in the vertical direction between the origin ‘O’ and the second point P2.

For example, the horizontal axis coordinate of a third point P3 may be 13, which is the number of pixels in the horizontal direction between the origin ‘O’ and the third point P3. The vertical axis coordinate of the third point P3 may be ‘3’, which is the number of pixels in the vertical direction between the origin ‘O’ and the third point P3.

For example, the horizontal axis coordinate of a fourth point P4 may be 16, which is the number of pixels in the horizontal direction between the origin ‘O’ and the fourth point P4. The vertical axis coordinate of the fourth point P4 may be ‘3’, which is the number of pixels in the vertical direction between the origin ‘O’ and the fourth point P4.

Referring to FIG. 5, the blob region information is illustrated as including the first to fourth points P1 to P4, but the scope of the present disclosure is not limited thereto.

The feature parameter generator may generate the feature parameter information corresponding to the time component of the abnormal breathing sound by applying the abscissa coordinate values of the blob region information to the spectrogram data.

In some embodiments, the size of the spectrogram data and the size of the heatmap data may be the same.

In some embodiments, the feature parameter generator may calculate the plurality of parameters indicating the time component based on the abscissa coordinate values of the blob region information and the reference time length. The reference time length may indicate the time length of one pixel of the spectrogram data. The reference time length may be determined by Equation 1.

$${t}_{p} = \frac{{S}_{rate}}{{L}_{hop}} \qquad [ Equation 1 ]$$

Equation 1 may be used to determine the reference time length. Here, tₚ refers to the reference time length, Sᵣₐₜₑ refers to the sampling rate of the breathing sound data, and Lₕₒₚ refers to the hop length of the spectrogram data.

The sampling rate of breathing sound data may indicate the number of sampling times per unit time when the sound sensor generates the breathing sound data from the user's breathing. The hop length of the spectrogram data may determine the size of data included in one pixel of the spectrogram data based on the size of the breathing sound data.

A plurality of parameters indicating the time component may be determined by Equation 2.

tₓ=bₓ×tₚ [Equation 2]

Equation 2 may be used to determine the plurality of parameters indicating the time component. Here, tₚ indicates the reference time length, bₓ may indicate one of the abscissa coordinate values, and tₓ may indicate one of the plurality of parameters indicating the time component.

For example, the start time of the blob region information may be calculated by multiplying 13, which is the abscissa coordinate value of the first point P1 or the third point P3, by the reference time length. The end time of a first blob region may be calculated by multiplying the abscissa coordinate value 16 of the second point P2 or the fourth point P4 by the reference time length.

The feature parameter generator may generate the feature parameter information corresponding to the frequency component of the abnormal breathing sound by applying the vertical axis coordinate values of the blob region information to the spectrogram data.

In some embodiments, the feature parameter generator may calculate the plurality of parameters indicating the frequency component based on the vertical axis coordinate values of the blob region information and the reference frequency magnitude. The reference frequency size may indicate the frequency magnitude of one pixel of the spectrogram data. The reference frequency magnitude may be determined by Equation 3.

$${f}_{p} = \frac{{S}_{rate}}{{N}_{f}} \qquad [ Equation 3 ]$$

Equation 3 may be used to determine the reference frequency magnitude. Here, fₚ may refer to the reference frequency magnitude, Sᵣₐₜₑ may refer to the sampling rate of the breathing sound data, and N_{f} may refer to the number of reference samples.

The number of the reference samples may indicate the number of breathing sound samples to be referenced per conversion operation when generating the spectrogram data from the breathing sound data.

The plurality of parameters indicating the frequency components may be determined by Equation 4.

f_{y}=b_{y}×fₚ [Equation 4]

Equation 4 may be used to determine the plurality of parameters indicating the frequency components. Here, fₚ may refer to the reference frequency magnitude, b_{y} may refer to one of the vertical axis coordinate values, and f_{y} may refer to one of the plurality of parameters indicating the frequency components.

For example, the lowest frequency of the blob region information may be calculated by multiplying ‘0’, which is the ordinate coordinate value of the first point P1 or the second point P2, by the reference frequency magnitude. For example, the highest frequency of the blob region information may be calculated by multiplying ‘3’, which is the ordinate coordinate value of the third point P3 or the fourth point P4, by the reference frequency magnitude.

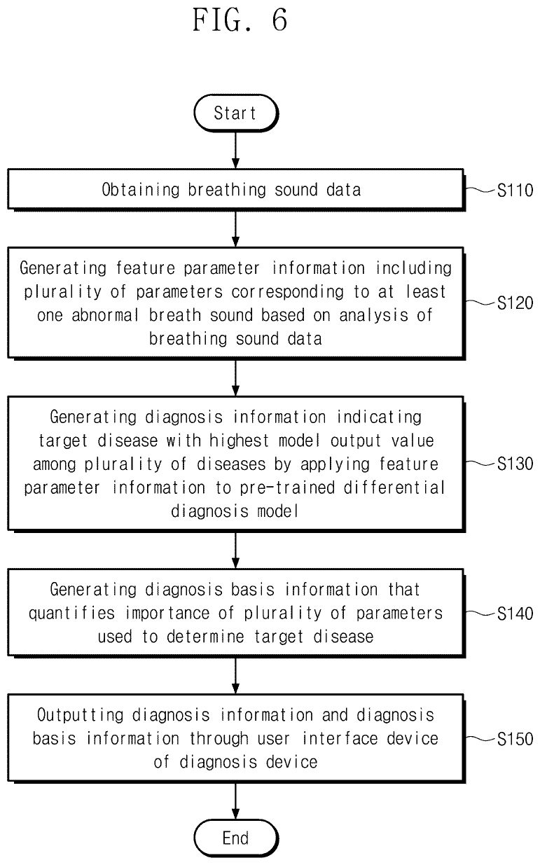

FIG. 6 is a flowchart describing a method of operating a diagnosis device according to some embodiments of the present disclosure. Referring to FIG. 6, a method of operating the diagnosis device will be described. The diagnosis device in FIG. 6 may correspond to the diagnosis device 100 in FIG. 1.

In operation S110, the diagnosis device may obtain breathing sound data by measuring the user's breathing.

In operation S120, the diagnosis device may generate feature parameter information including a plurality of parameters corresponding to at least one abnormal breath sound based on analysis of breathing sound data.

In some embodiments, operation S120 may include generating spectrogram data that visually represents the time and frequency components of at least one abnormal breath sound based on the breathing sound data, generating heatmap data including pixels each having a temperature value according to the probability of corresponding to at least one abnormal breathing sound based on the convolution neural network operation on the spectrogram data, and generating feature parameter information based on the spectrogram data and the heatmap data.

In some embodiments, the size of the spectrogram data and the size of the heatmap data may be the same.

In some embodiments, operation S120 may further include detecting a blob region by filtering the heatmap data based on a threshold temperature value, extracting coordinates of the blob region from the heatmap data, and generating feature parameter information corresponding to the time component and frequency component of at least one abnormal breathing sound based on the spectrogram and the extracted coordinates.

In some embodiments, operation S120 may further include generating feature parameter information corresponding to the time component and frequency component of at least one abnormal breathing sound by overlapping the heatmap data and the spectrogram data, i.e., by applying coordinates extracted from the heatmap data to the spectrogram data.

In operation S130, the diagnosis device may generate diagnosis information indicating the target disease with the highest model output value among the plurality of diseases by applying the feature parameter information to a pre-trained differential diagnosis model.

In operation S140, the diagnosis device may generate diagnosis basis information that quantifies the importance of the plurality of parameters used to determine the target disease.

In operation S150, the diagnosis device may output the diagnosis information and the diagnosis basis information through the user interface device of the diagnosis device.

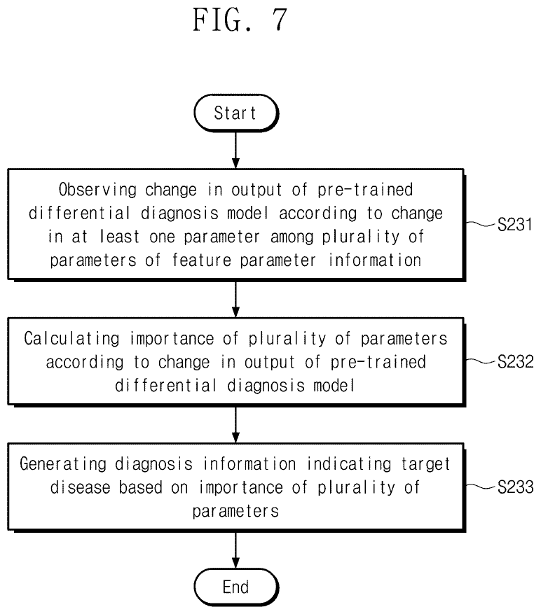

FIG. 7 is a flowchart describing a method of generating diagnosis information by a differential diagnosis device according to some embodiments of the present disclosure. Referring to FIG. 7, a method by which a differential diagnosis device generates diagnosis information will be described. The differential diagnosis device of FIG. 7 may correspond to the differential diagnosis device 140 of FIG. 1.

In operation S231, the differential diagnosis device may observe a change in the output of the pre-trained differential diagnosis model according to a change in at least one parameter among the plurality of parameters of the feature parameter information.

In some embodiments, operation S231 may further include comparing the output of the pre-trained differential diagnosis model DDM when one parameter among the plurality of parameters is present with the output of the pre-trained differential diagnosis model DDM when one parameter among the plurality of parameters is absent.

In operation S232, the differential diagnosis device may calculate the importance of the plurality of parameters according to changes in the output of the pre-trained differential diagnosis model.

In operation S233, the differential diagnosis device 140 may generate the diagnosis information indicating the target disease based on the importance of the plurality of parameters.

According to an embodiment of the present disclosure, a diagnosis device that provides diagnosis information and diagnosis basis information and a method of operating the same are provided.

In addition, according to an embodiment of the present disclosure, a diagnosis device that improves user convenience and increases usability and a method of operating the same are provided by generating not only diagnosis information indicating the classified disease, but also multiple parameters indicating features of abnormal breathing sounds, and by providing the user with diagnosis basis information that quantifies the importance of each of the plurality of parameters used to classify the disease. a spike neural network circuit including double precision asynchronous neurons and a method of operating the same are provided.

The above descriptions are specific embodiments for carrying out the present disclosure. Embodiments in which a design is changed simply or which are easily changed may be included in the present disclosure as well as an embodiment described above. In addition, technologies that are easily changed and implemented by using the above embodiments may be included in the present disclosure. Therefore, the scope of the present disclosure should not be limited to the above-described embodiments and should be defined by not only the claims to be described later, but also those equivalent to the claims of the present disclosure.

What is claimed is:

1. A method of operating a diagnosis device for diagnosing a disease of a user, the method comprising:
obtaining breathing sound data by measuring a breath of the user;
generating feature parameter information including a plurality of parameters corresponding to at least one abnormal breathing sound based on an analysis of the breathing sound data;
generating diagnosis information indicating a target disease with a highest model output value among a plurality of diseases by applying the feature parameter information to a pre-trained differential diagnosis model;
generating diagnosis basis information that quantifies an importance of the plurality of parameters used to determine the target disease; and
outputting the diagnosis information and the diagnosis basis information through a user interface device of the diagnosis device.

2. The method of claim 1, wherein the generating of the diagnosis information indicating a target disease with the highest model output value among the plurality of diseases by applying the feature parameter information to the pre-trained differential diagnosis model includes;
observing a change in an output of the pre-trained differential diagnosis model depending on a change in at least one parameter among the plurality of parameters of the feature parameter information;
calculating the importance of the plurality of parameters depending on the change in the output of the pre-trained differential diagnosis model; and
generating the diagnosis information indicating the target disease based on the importance of the plurality of parameters.

3. The method of claim 2, wherein the importance of the plurality of parameters indicates a priority of each of the plurality of parameters with respect to the target disease.

4. The method of claim 1, wherein the plurality of parameters of the feature parameter information include at least one of the number of occurrences, occurrence intensity, start time, end time, duration, lowest frequency, maximum frequency, and average frequency of the at least one abnormal breathing sound.

5. The method of claim 1, wherein the generating of the feature parameter information including the plurality of parameters corresponding to the at least one abnormal breathing sound based on the analysis of the breathing sound data includes:
generating spectrogram data that visually represents a time component and a frequency component of the at least one abnormal breathing sound based on the breathing sound data;
generating heatmap data including pixels each having a temperature value depending on a probability of corresponding to the at least one abnormal breathing sound based on a convolution neural network operation of the spectrogram data; and
generating the feature parameter information based on the spectrogram data and the heatmap data.

6. The method of claim 5, wherein the generating of the feature parameter information based on the spectrogram data and the heatmap data includes:
detecting a blob region by filtering the heatmap data based on a threshold temperature value;
extracting coordinates of the blob region from the heatmap data; and
generating the feature parameter information corresponding to the time component and the frequency component of the at least one abnormal breathing sound based on the spectrogram data and the extracted coordinates.

7. The method of claim 6, wherein the generating of the feature parameter information corresponding to the time component and the frequency component of the at least one abnormal breathing sound based on the spectrogram data and the extracted coordinates includes:
calculating the plurality of parameters corresponding to the time component based on abscissa coordinate values of the extracted coordinates and a reference time length, and
wherein the reference time length is determined based on equation
$${t}_{p} = \frac{{S}_{rate}}{{L}_{hop}} ,$$
where tₚ refers to the reference time length, Sᵣₐₜₑ refers to a sampling rate of the breathing sound data, and Lₕₒₚ refers to a hop length of the spectrogram data, and
wherein the plurality of parameters corresponding to the time component are determined based on equation tₓ=bₓ×tₚ, where tₚ indicates the reference time length, bₓ indicates one of the abscissa coordinate values, and tₓ indicates one of the plurality of parameters corresponding to the time component.

8. The method of claim 6, wherein the generating of the feature parameter information corresponding to the time component and the frequency component of the at least one abnormal breathing sound based on the spectrogram data and the extracted coordinates includes:
calculating a plurality of parameters corresponding to the frequency component based on ordinate coordinate values of the extracted coordinates and a reference frequency magnitude, and
wherein the reference frequency magnitude is determined based on equation
$${f}_{p} = \frac{{S}_{rate}}{{N}_{f}} ,$$
where fₚ refers to the reference frequency magnitude, Sᵣₐₜₑ refers to a sampling rate of the breathing sound data, and N_{f} refers to the number of reference samples, and
wherein the plurality of parameters corresponding to the frequency component are determined based on equation by f_{y}=b_{y}×fₚ, where fₚ indicates the reference frequency magnitude, b_{y} indicates one of the ordinate coordinate values, and f_{y} indicates one of the plurality of parameters corresponding to the frequency component.

9. The method of claim 5, wherein the generating of the heatmap data including pixels each having the temperature value depending on the probability of corresponding to the at least one abnormal breathing sound based on a convolution neural network operation of the spectrogram data includes:
generating the heatmap data from the spectrogram data based on a top-down method.

10. A diagnosis device comprising:
a sound sensor configured to measure a breath of a user to generate breathing sound data;
a breathing sound analysis device configured to generate spectrogram data that visually represents a time component and a frequency component of at least one abnormal breathing sound based on an analysis of the breathing sound data, and to generate heatmap data including pixels each having a temperature value depending on a probability of corresponding to the at least one abnormal breathing sound, based on a convolution neural network operation of the spectrogram data;
a feature parameter extraction device configured to generate feature parameter information including a plurality of parameters corresponding to the at least one abnormal breathing sound based on the spectrogram data and the heatmap data;
a differential diagnosis device configured to generate diagnosis information indicating a target disease with a highest model output value among a plurality of diseases by applying the feature parameter information to a pre-trained differential diagnosis model, and to generate diagnosis basis information that quantifies an importance of the plurality of parameters used to determine the target disease; and
a user interface device configured to output the diagnosis information and the diagnosis basis information.

11. The diagnosis device claim 10, wherein a breathing sound analysis device includes:
a spectrogram generator configured to receive the breathing sound data from the sound sensor and to generate the spectrogram data from the breathing sound data; and
an abnormal breathing sound detection device configured to receive the spectrogram data from the spectrogram generator and to generate the heatmap data from the spectrogram data.

12. The diagnosis device claim 11, wherein the feature parameter extraction device includes:
a blob region detector configured to receive the heatmap data from the abnormal breathing sound detection device, to detect a blob region by filtering the heatmap data based on a threshold temperature value, to extract coordinates of the blob region from the heatmap data, and to generate blob region information indicating the extracted coordinates; and
a feature parameter generator configured to receive the spectrogram data from the spectrogram generator, to receive the blob region information from the blob region detector, and to generate the feature parameter information corresponding to the time component and the frequency component of the at least one abnormal breathing sound, based on the spectrogram data and the blob region information.

13. The diagnosis device claim 10, wherein the differential diagnosis device includes:
a diagnosis information generator configured to receive the feature parameter information from the feature parameter extraction device, to observe a change in an output of the pre-trained differential diagnosis model depending on a change in at least one parameter among the plurality of parameters of the feature parameter information, to calculate the importance of the plurality of parameters depending on the change in the output of the pre-trained differential diagnosis model, and to generate the diagnosis information indicating the target disease based on the importance of the plurality of parameters; and
a diagnosis basis information generator configured to generate the diagnosis basis information that quantifies the importance of the plurality of parameters used to determine the target disease.
